(19) United States
(12) Patent Application Publication
(10) Pub. No.: US 2024/0216015 A1
(43) Pub. Date: Jul. 4, 2024

(54) SYSTEM AND METHOD FOR INSERTING AND REMOVING AN EXPANDABLE DEVICE IN A BODY CAVITY

(71) Applicant: Innoventions Ltd., Or Akiva (IL)

(72) Inventors: Daniel Yachia, Herzliya (IL); Ofir Yulish, Yahud Monsun (IL); Yehonatan Mathis, Binyamina (IL)

(73) Assignee: Innoventions Ltd., Or Akiva (IL)

(21) Appl. No.: 18/090,093

(22) Filed: Dec. 28, 2022

(51) Int. Cl. A61B 17/34; A61B 17/12

(52) U.S. Cl. CPC A61B 17/3468; A61B 17/12031; A61B 17/12136; A61B 2017/00641

(57) ABSTRACT

An applicator for delivering a fillable balloon into a body cavity. The applicator has a tubular shaft and a tubular capsule attached or adapted to be attached to a distal end of the shaft. The capsule receives a balloon in an uninflated or unfilled or partially filled state. A pusher adapted to be inserted into a valve of the balloon serves to push the balloon out from the capsule lumen, conduct a fluid to the balloon to inflate or fill the balloon with the fluid, and detach the balloon from the distal end of the pusher

### FIELD OF THE INVENTION

The present invention relates to medical devices, and more specifically to such devices for inserting and removing an object into and from a body cavity.

### BACKGROUND OF THE INVENTION

There are many cases where it is desirable to insert an inflatable or expandable device, such as a balloon, into a body cavity or lumen. Systems for inserting a balloon into a body cavity include an inserter configured to retain the balloon in a collapsed, unfilled or partially filled configuration at the distal end of the applicator. After delivery of the distal end of the inserter to the body cavity to be treated, the balloon is filled with a fluid and is then released from the inserter. The inserter is then removed from the body leaving the filled or expanded balloon in the body cavity.

U.S. Pat. No. 6,293,923 describes a system for inserting a fillable or expandable balloon into a urinary bladder in the control of urinary incontinence or for the release of medicaments into the bladder. U.S. Pat. No. 11,179,257 describes a system for delivering an expandable balloon into the stomach for the treatment of obesity. International Patent Publication WO2017/025959 (also published as U.S. Pat. No. 10,631,966 describes inserting a balloon into a body cavity in the treatment of an unwanted fistula.

A “vesicovaginal fistula” is a fistula or passageway between the urinary bladder and the vagina and is the most common urogenital fistula. This fistula allows constant leakage of urine form the bladder into the vagina and out from the vagina, and results in frequent vaginal and vulvar irritation and bladder infections.

Vesicovaginal fistulas are usually treated by surgical repair. The odor from constant leakage, combined with misperceptions about its cause, can result in stigma and ostracism. Many women with fistula are abandoned by their husbands and families. They may find it difficult to secure income or support, thereby deepening their poverty. In the developed world, these fistulas are uncommon and usually result from complications of gynecological surgery, pelvic abscess or pelvic irradiation. In poor developing countries, however, these fistulas are more common and are related to obstructed labor due to unattended deliveries, small pelvic dimensions, malpresentation, poor uterine contractions and introital stenosis, especially in very young girls. In some parts of Africa, it is estimated that in as many as 3-4 per 1000 vaginal deliveries women develop these fistulas.

There are no non-surgical treatments for vesicovaginal fistulas. Rarely, a very small vesicovaginal may close spontaneously. Most vesicovaginal fistulas require surgery to close the opening usually through the abdomen or vagina.

### SUMMARY OF THE INVENTION

In one of its aspects, the present invention provides a system for inserting an expandable balloon into a body cavity. The system of the invention may be used, for example, to deliver an expandable balloon into the urinary bladder rectum, or stomach.

The system of the invention comprises a fillable balloon configured to be inserted into the body cavity. The system also comprises an insertion applicator for delivering the fillable balloon into the body cavity to, for example, to occlude a fistula opening. The insertion applicator is configured for filling the balloon and releasing the filled or expandable balloon into the body cavity. A tubular capsule is attached or attachable to the distal end of a shaft of the inserter and configured to receive in its interior the balloon in the unfilled or partially filled configuration. The applicator further includes a pusher comprising a small caliber tube along the applicator shaft. The distal end of the tube contains an injection needle which is inserted into a filling valve in the wall of the balloon before the balloon is folded or crumpled into the capsule. The pusher and the needle not only serve to push the unfilled or partially filled balloon from the capsule and but also to deliver a filling fluid or gas rom a source located at the proximal end of the inserter to fill the balloon. After filling of the balloon is completed, the balloon is detached from the distal end of the pusher and released into the body cavity. The shaft of the inserter ean is then removed from the body, leaving the filled balloon in the body cavity.

The system of the invention may be used, for example, to insert a fillable or inflatable balloon into the urinary bladder in the treatment of urinary incontinence or other bladder treatments, or into the stomach in the treatment of obesity.

The system of the invention may also be used to deliver a fillable or inflatable balloon into a urinary bladder to occlude a vesicorectal or vesicovaginal fistula.

Thus, in one of its aspects, the invention provides an applicator for delivering a fillable balloon into a body cavity comprising:
(a) a tubular shaft having a lumen;
(b) a tubular capsule attached or adapted to be attached to a distal end of the shaft; the capsule having a lumen adapted to receive a balloon in an uninflated or unfilled or partially filled state; and
(c) a pusher located in a lumen of the tubular shaft having a distal end adapted to be inserted into a valve of the balloon, the pusher being adapted to
   push the balloon out from the capsule lumen;
   conduct a fluid from a proximal end of the applicator to the balloon to inflate or fill the balloon with the fluid through the valve; and
   detach the balloon from the distal end of the pusher

In the applicator of the invention, the shaft may have a slit in a wall of the shaft, exposing the lumen of the shaft to the exterior.

The pusher may include a hollow tube with a needle at its distal end. The pusher may be connected to a male Luer connector at the proximal end of the pusher.

The capsule may be adapted to be attached to the shaft and to lock onto the distal end of the shaft.

The applicator may be adapted to deliver a fillable balloon to a body cavity selected from urinary bladder, a rectum, a stomach or a vagina.

The capsule may be straight or curved.

In another of its aspects, the invention provides a balloon having a magnet for use in the applicator of the invention.

In another of its aspects, the invention provides a capsule for use in the applicator of the invention, the capsule having a fillable balloon in an unfilled state folded, rolled or crumpled in the interior of the capsule.

The capsule may be straight, curved or bent. The balloon may have a magnet.

In yet another of its aspects, the invention provides a system for delivering a fillable balloon into a body cavity comprising:
(a) an applicator according to claim 1; and
(b) one or more balloons configured to be folded or crumpled in the lumen of the capsule of the applicator;

The system may further comprise a retrieval device configured to deflate a filled balloon in the body cavity and to remove the balloon from the body cavity. The retrieval device may comprise:
(a) a needle that is movable between a retracted position, and an extended position;
(b) a plunger attached to the needle, the plunger being spring biased by a spring in the extended position, and
(c) a locking mechanism allowing the needle to be intermittently locked in the retracted position.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1a shows an assembled view and FIG. 1b shows an exploded view of an insertion device that may be used to insert a fillable balloon into a urinary bladder of a female subject, in accordance with one embodiment of the invention;

FIG. 2a shows a top view and FIG. 2b shows a side view of a balloon that may be used to occlude the urinary bladder side of a vesico-vaginal fistula in accordance with one embodiment of the invention;

FIG. 2c shows a top view and FIG. 2d shows a perspective view of another balloon that may be used to occlude the urinary bladder side of a vesico-vaginal fistula in accordance with another embodiment of the invention;

FIG. 3a depicts a fillable balloon rolled-up in the capsule of the insertion device of FIG. 1a in a longitudinal section. FIG. 3b depicts the balloon in a cross section. FIG. 3c depicts the fillable balloon crumpled in the capsule of the insertion device of FIG. 2a in a longitudinal section. FIG. 3d depicts the crumpled balloon in a cross section.

FIG. 4 shows a vesico-vaginal fistula a utero-vesical fistula and a rectovaginal fistula in a female subject;

FIGS. 5a to 5f show a process of insertion of a balloon into a urinary bladder of a female subject using the insertion device of FIG. 1;

FIG. 6a shows an insertion device having a straight capsule for inserting a balloon into a urinary bladder of a male subject in accordance with one embodiment of the invention;

FIG. 6b shows an insertion device having a curved capsule for inserting a balloon into a urinary bladder of a male subject in accordance with one embodiment of the invention;

FIG. 7a shows the insertion device of FIG. 6a after insertion through the urethra into the urinary bladder of a male subject;

FIG. 7b shows the insertion device of FIG. 6b after insertion through the urethra into the urinary bladder of a male subject; and

FIG. 8a depicts a retrieval device for removing a balloon from a body cavity of a female or male subject, having a movable needle in a retracted position. FIG. 8b depicts the extraction device with the needle in an extended position.

### DESCRIPTION OF THE INVENTION

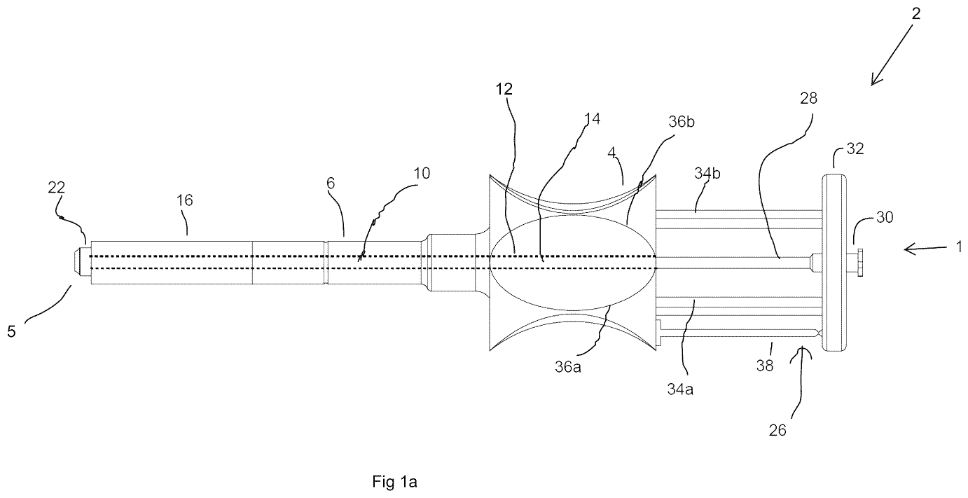

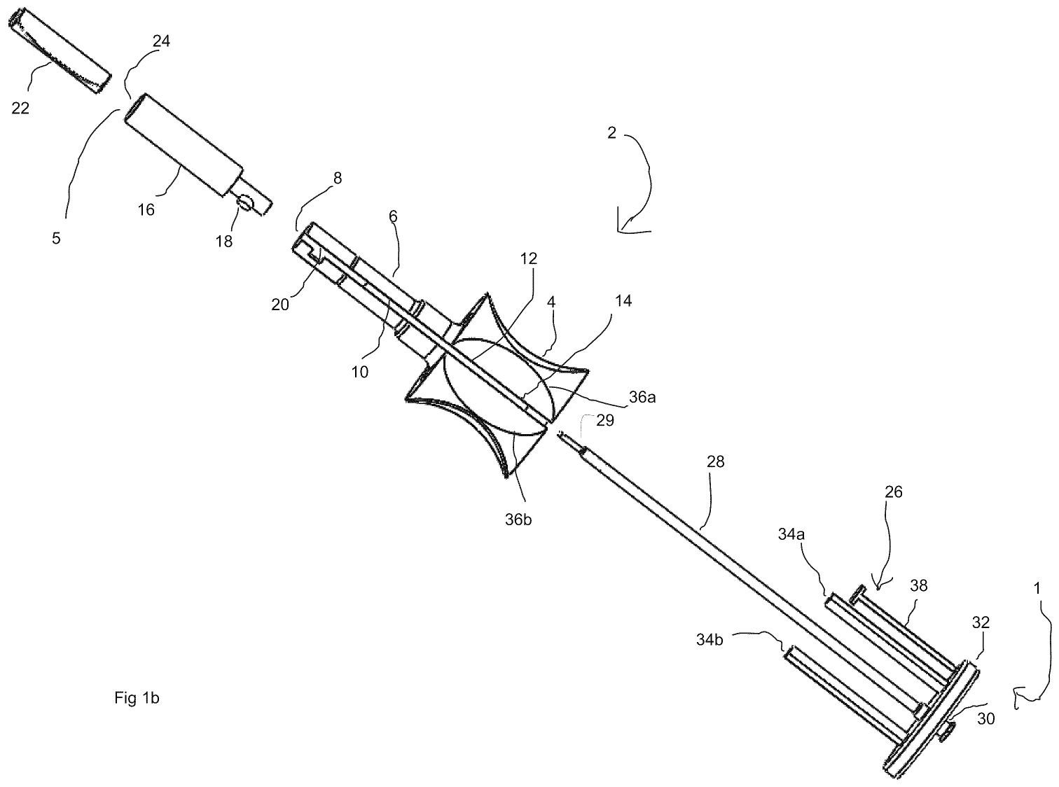

FIG. 1 shows an insertion applicator 2 for delivering a fillable balloon into a urinary bladder of a female subject in accordance with one embodiment of the invention. The insertion applicator 2 is shown in an assembled view in FIGS. 1a and 1n an exploded view in FIG. 1b, and has a proximal end 1 and a distal end 5. The applicator 2 has a gripping element 4 attached to a tubular shaft 6. The shaft may have, although not necessarily, an incomplete cylindrical wall so that a lumen 8 of the tubular extension is exposed to the exterior through a slit 10 in the wall of the shaft. The lumen 8 of the shaft is aligned with a lumen 12 in the interior of the gripping element 4. The lumen 12 of the gripping element 4 may also be exposed to the exterior through a slit 14 in the gripping element 4. The tubular shaft 6 can be integral with the gripping element 4. A tubular capsule 16 is adapted to be attached to the shaft 6 and to lock onto the distal end of the shaft 6 by means of a knob 18 that is received in a notch 20 near the distal end of the shaft (FIG. 1b). The capsule 16 is configured to receive into a lumen 24 of the capsule 16, an inflatable balloon in an uninflated, partially filled or unfilled state. The applicator 2 further includes a pusher 32 that is used to push the balloon out from the capsule 16 prior to filling or inflating the balloon, as explained below. The pusher includes a tube 28 and a hollow needle 29 that is used to hold the folded balloon 22 in the capsule and to fill the balloon with a fluid after the balloon is pushed out of the capsule, as explained below. The needle 29 and the tube 28 pass through a hole in the pusher 32 and is affixed to the pusher 32 near the proximal end of the tube 28. The proximal end of the tube is provided with a male Luer connector 30 at the proximal end that can be attached to a source of a pressurized fluid, such as a syringe, for filling the balloon.

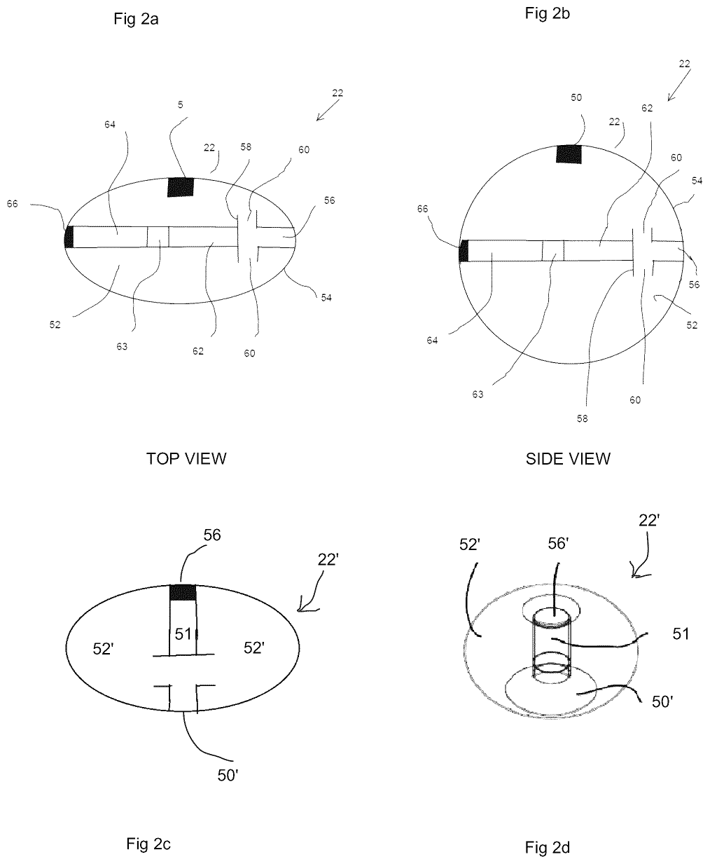

FIGS. 2a and 2b show one embodiment of a balloon 22 that may be used with the insertion applicator 2, for example, to occlude the bladder opening of a vesicovaginal or vesicorectal fistula. The balloon 22 is shown in FIGS. 2a and 2b in its expanded state after having been filled with a fluid. The expanded balloon 22 has a disc-like shape, and is shown in a top view in FIG. 2a and in a side view in FIG. 2b. The balloon 22 is provided with a magnet 50 located in the lumen cavity 52 of the balloon and attached to a wall 54 of the balloon 22. The balloon 22 also has a self-sealing valve 56 that is configured to receive the tip of the needle 28 through which a filling fluid is introduced into the cavity 52 of the balloon 22. In use, the tip of the needle 28 is inserted through the valve 56 until the tip of the needle 28 contacts a barrier 58 of the valve. Fluid extruded from the tip of the needle under pressure leaves the valve 56 through lateral vents 60, and enters the balloon cavity 52. The valve can be a duck-bill valve, a self-sealing valve or any other uni-directional valve. The balloon 22 further comprises a first strut 62 attached to the barrier 58, and a second strut 64 attached to an attachment site 66 diametrically opposed to the valve 56. The struts 62 and 64 are joined near the center of the balloon lumen 62 by a flexible connector that acts as a hinge.

FIGS. 2c and 2d show an alternative fillable balloon 22′ that may be used with the insertion applicator 2. The balloon 22′ is shown in a sectional drawing in FIG. 2c and in a perspective view in FIG. 2d. The balloon 22′ is provided with a magnet 50′ located in a central core 51 in the cavity 52′ of the balloon. The balloon 22′ also has a filling valve 56′ that is configured to receive the tip of the needle 28 through which a filling fluid is introduced into the cavity 52′ of the balloon 22′. The tip of the needle 28 is inserted through the valve 56′ and fluid extruded from the tip of the needle 28 under pressure leaves the valve 56′. and enters the balloon cavity 52′. The valve can be a duck-bill valve, a self-sealing valve or any other uni-directional valve.

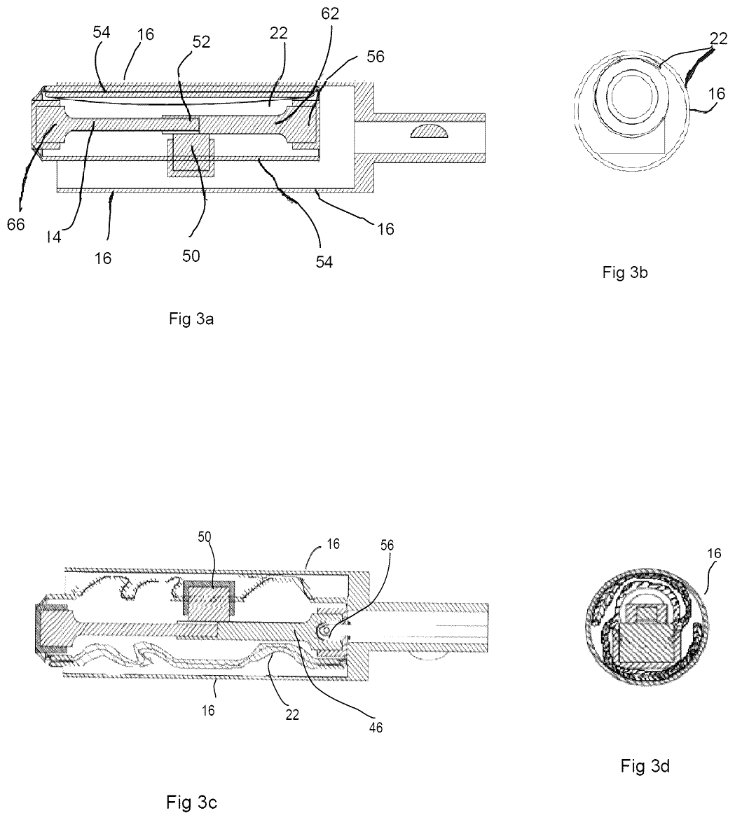

The unfilled or partially filled balloon 22 or 22′ can be rolled up and inserted into the capsule 16. FIG. 3 shows a longitudinal sectional view (FIG. 3a) and a cross-sectional view (FIG. 3b) of the capsule 16 showing the unfilled or partially filled balloon 22 or 22′ rolled up inside the cavity of the capsule 16. Alternatively, the unfilled or partially filled balloon may be crumpled up and inserted into the capsule 16. FIG. 3c shows a longitudinal sectional view and FIG. 3d shows a cross-sectional view of the capsule 16 showing the unfilled balloon 22 or 22′ crumpled up inside the cavity of the capsule 16.

Referring again to FIG. 1, the needle 29 and its attached tube 28 are slidable in the common lumen formed by the gripper 4, the shaft 6 and the capsule 16. In the assembled state (FIG. 1a), the tip of the needle 28 is positioned in the filling valve 56 or 56′ of the balloon 22 or 22′, respectively. The pusher 32 is provided with guides 34a and 34b that can be inserted into guide ways 36a and 36b, respectively, in the gripper 4, in order to guide movement of the needle. A stopper 38 prevents movement of the pusher 26 beyond the position shown in FIG. 1a during transport and storage of the applicator 2. The stopper 38 is removed prior to use in order to allow the needle to move distally with the empty or partially empty balloon 22 or 22′.

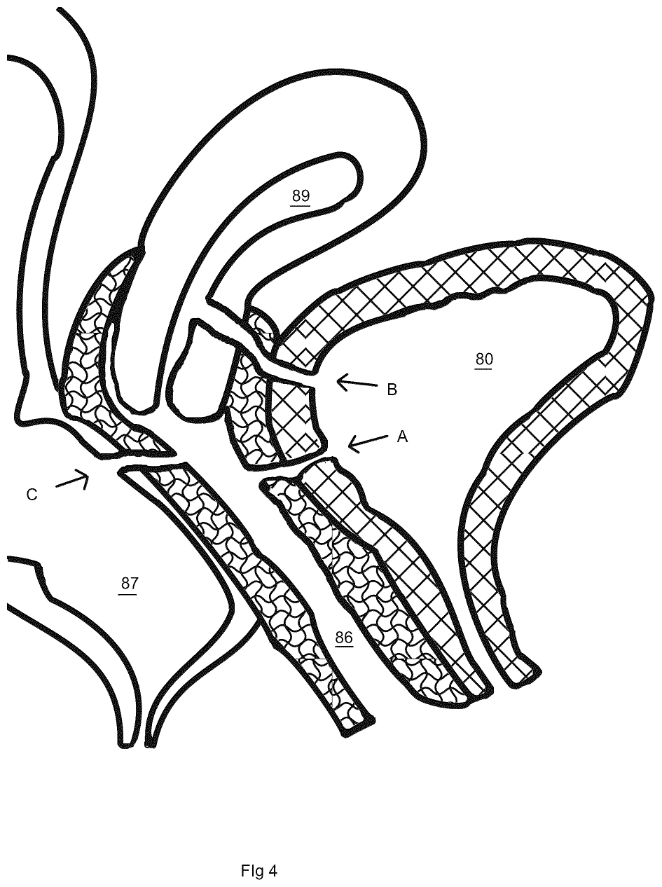

FIG. 4 shows three types of urogenital fistulas in a female subject. Arrow A indicates a fistula between the urinary bladder 80 and the vagina 86 (a vesicovaginal fistula). Arrow B indicates a fistula between the urinary bladder 80 and the uterus 89 (a utero-vesical fistula). Arrow C indicates a fistula between the vagina 86 and the rectum 87 (a rectovaginal fistula). These fistulas A, B and C form undesirable passageways for substances between the bladder 80, rectum 87 and the vagina 86.

Use of the applicator 2 will now be described in relation to deploying a balloon in a urinary bladder, for example, in the treatment of a vesicovaginal fistula. This is by way of example only, it being evident how the system of the invention can be used to deliver a balloon to any desired body cavity.

FIGS. Sa to 5f shows a sequence of steps in which the balloon 22 or 22′ is deployed in a urinary bladder 80 for occluding the bladder side opening 82 of a vesicovaginal fistula 84.

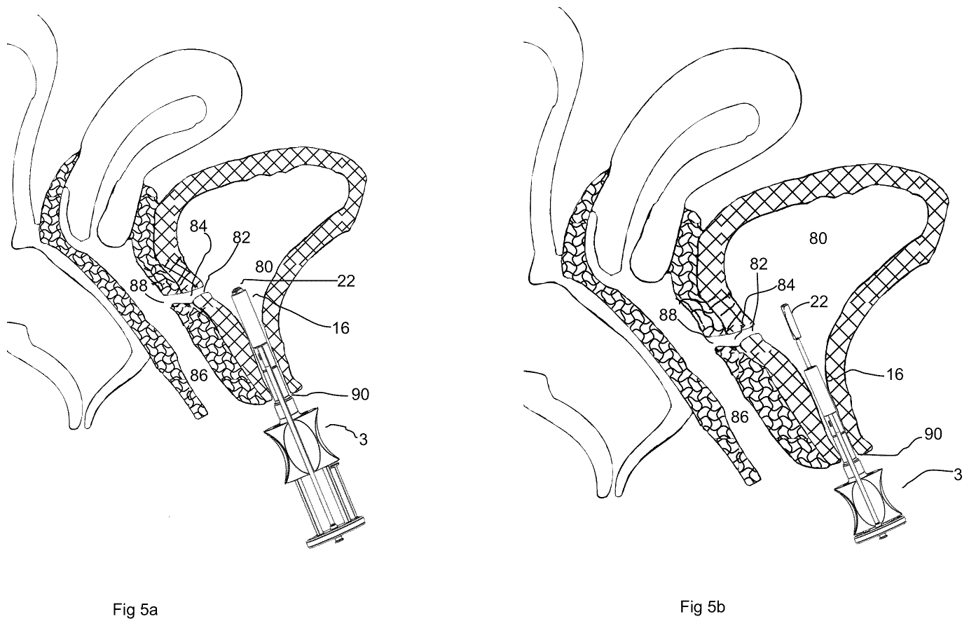

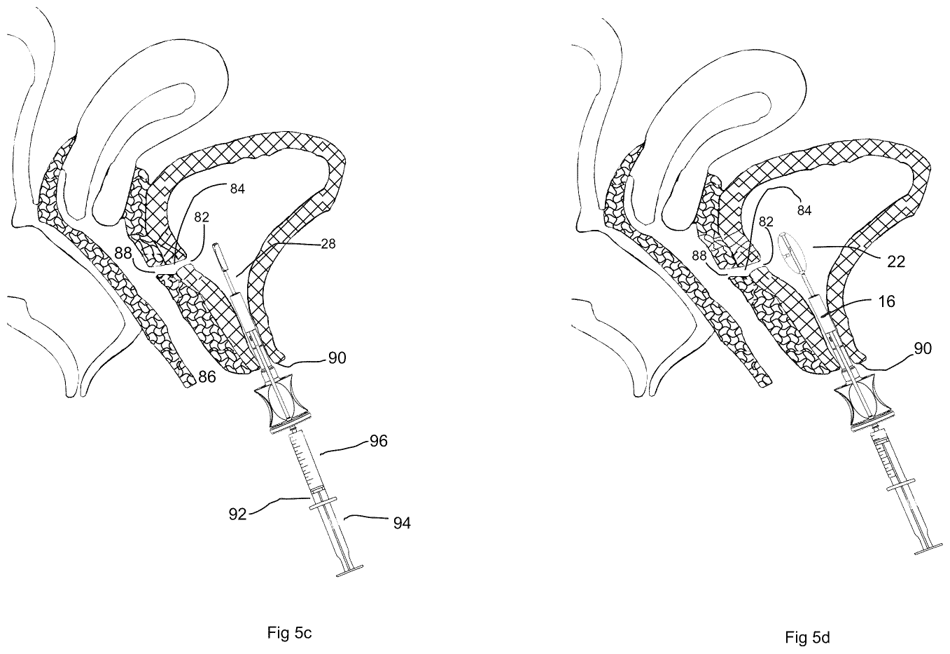

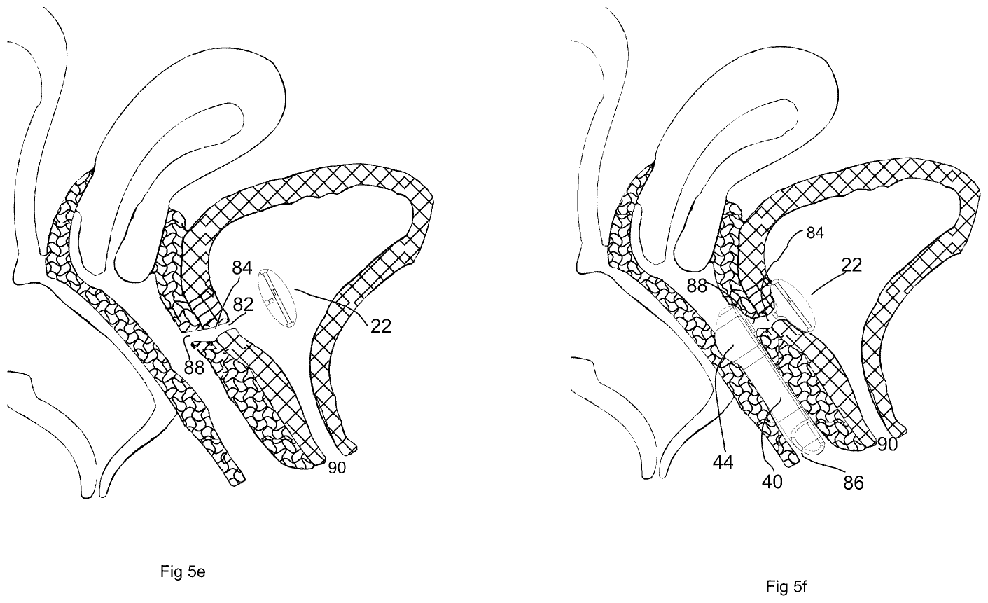

As shown in FIG. 5a, after loading the capsule 16 with a balloon such as the balloon 22 or 22′, the distal end of the insertion applicator 2 is inserted through the urethra 90 until the capsule 16 is positioned in the bladder 80. At this point, the stopper 38 is removed from the pusher 26 and the pusher 26 is pushed distally until the balloon 22 or 22′ has been completely pushed out from the capsule 16 while remaining connected to the needle (FIG. 5b).

After pushing the balloon 22 or 22′ out from the capsule 16, a syringe 92, or other source of pressurized fluid, is attached to the Luer fitting 30 (FIG. 5c), and the piston 94 of the syringe 92 is depressed to force fluid in the barrel 96 of the syringe 92 through the needle and into the lumen 52 of the balloon. This brings the balloon into its filled state (FIG. 5d). Pulling the needle 28 proximally brings the inflated balloon into contact with the distal end of the capsule 16, and further pulling of the needle or the entire inserting applicator causes the balloon 22 to detach from the needle and to be released from the applicator 2 to float in the bladder 80. The applicator 2 can then be removed from the body (FIG. 4e).

A vaginal element 40 containing an internal magnet 50 (FIG. 5f) is inserted into the vagina 86 that becomes lodged in the vagina 86. The vaginal element 40 may have a bulge 42 at the level of the internal magnet 44. The polarity of the magnet 44 in the vaginal plug 40 and the polarity of the magnet 50 or 50′ in the balloon 22 or 22′, respectively, are determined so that the magnet 50 or 50′ is attracted to the magnet 44, which brings the balloon 22 or 22′ into position to occlude the bladder opening 82 of the fistula 84 (FIG. 5f), while the vaginal element 40 occludes the vaginal opening 88 of the fistula 84.

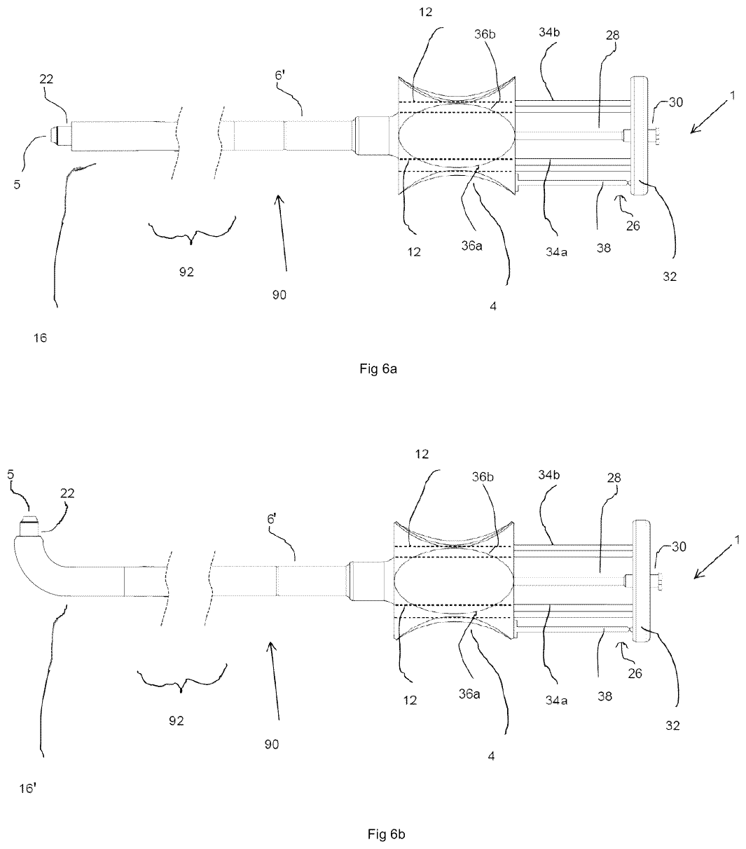

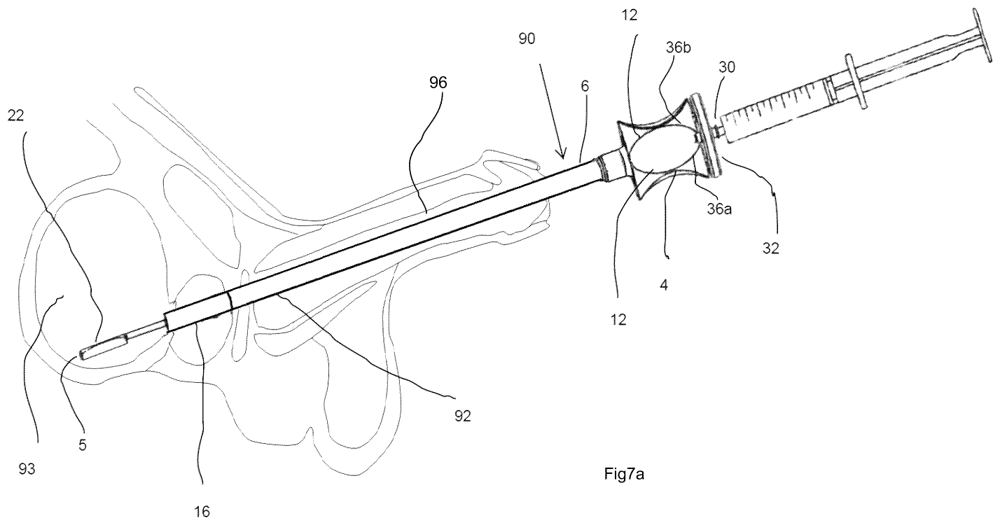

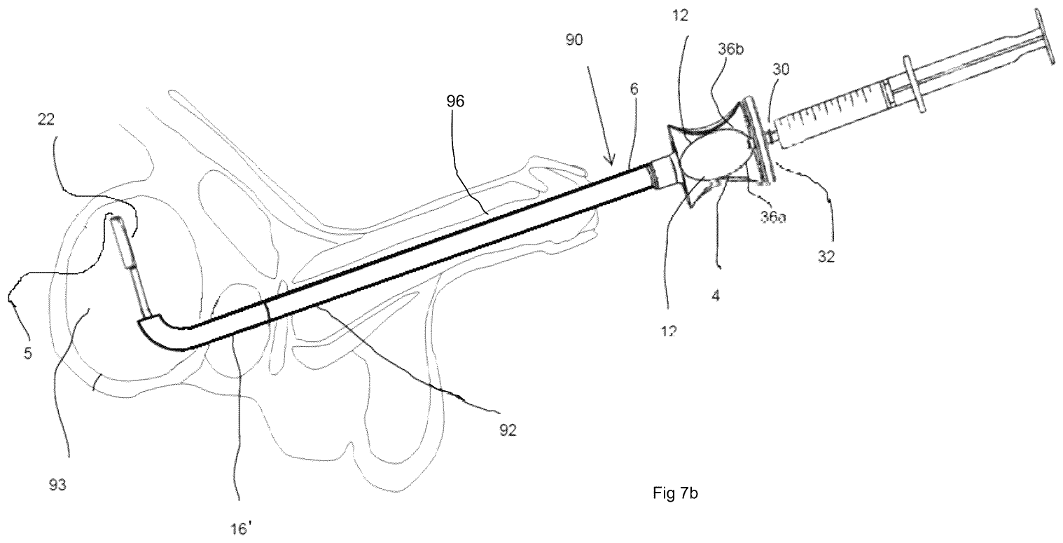

FIG. 6a shows an applicator 90 for delivering a balloon, such as the balloon 22 or 22′ into the urinary bladder of a male subject, accordance with one embodiment. The applicator 90 has several components in common with the applicator 2 shown in FIG. 2, and similar components are indicated in FIG. 6 with the same reference numerals used in FIGS. 2a and 2b. The shaft 6′ of the delivery device 90 is longer than the shaft 6 of the female delivery device 2 due to the longer length of the male urethra. FIG. 6b shows an alternative embodiment 91 of an applicator for delivering a balloon in the urinary bladder of a male subject. In the delivery device 91, the capsule 16′ is curved in order to facilitate delivery of the balloon into the male urinary bladder. FIG. 7a shows the delivery device 90 after insertion through the urethra 96 into the urinary bladder 93 of a male subject and release of the balloon 22 from the capsule 16. FIG. 7b shows the delivery device 91 after insertion through the urethra 96 into the urinary bladder 93 of a male subject and release of the balloon 22 from the capsule 16.

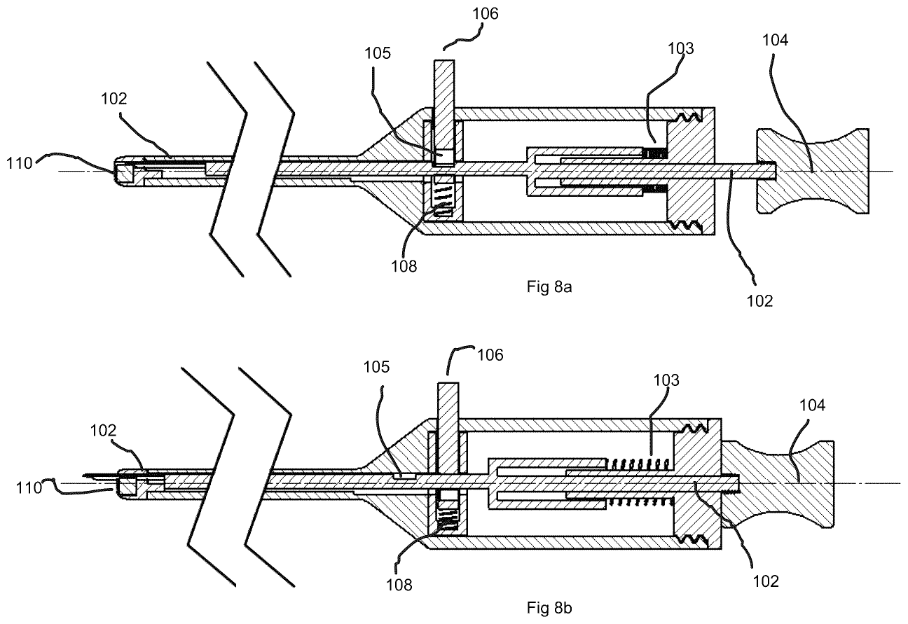

FIGS. 8a and 8b show a retrieval device 100 that may be used to retrieve the balloon 22 or 22′ when it is desirable to remove the balloon from the body. The retrieval device 100 has a magnet 100 at the tip of the shaft to engage the magnet in the balloon It also has a needle 102 that is movable between a retracted position, shown in FIG. 8a and an extended position shown in FIG. 8b. The needle 102 is attached to a plunger 104 that is spring biased by a spring 103 to maintain the needle in the extended position. A notch 105 on the side of the plunger 104 allows the needle 102 to be locked in the retracted position, against the action of the spring 104, by means of a bolt 106 that is spring biased in a locking position by means of a spring 108. In the locking position, shown in FIG. 8a, the bolt 106 is inserted into the notch 105. Sliding of the bolt 106 from the locking position, against the spring 108 causes the shaft to slide distally under the influence of the spring 104 to bring the needle into the extended position (FIG. 8b).

In practice, the vaginal element 40 (FIG. 5f) is removed from the vagina to allow the balloon to float in the bladder. The distal end of the retriever is inserted through the urethra into the urinary bladder with the needle 102 locked in the retracted position. After insertion into the urinary bladder, the magnet 100 at the tip of the retriever engages the magnet of the balloon. Then the needle 102 is brought to the extended position by movement of the bolt 106 out of the notch 105. The needle 102 pierces the balloon 22, to allow release of the filling fluid from the balloon 22. The magnet 110 at the distal end of the shaft of the retriever which engaged the magnet of the balloon 22 together with the balloon 22 can be withdrawn from the bladder together with retrieval device.

1. An applicator for delivering a fillable balloon into a body cavity comprising:
(a) a tubular shaft having a lumen;
(b) a tubular capsule attached or adapted to be attached to a distal end of the shaft; the capsule having a lumen adapted to receive a balloon in an uninflated or unfilled or partially filled state;
and,
(c) a pusher located in a lumen of the tubular shaft having a distal end adapted to be inserted into a valve of the balloon, the pusher being adapted to:
push the balloon out from the capsule lumen;
conduct a fluid from a proximal end of the applicator to the balloon to inflate or fill the balloon with the fluid through the valve; and,
detach the balloon from the distal end of the pusher.

2. The applicator according to claim 1, wherein the shaft has a slit in a wall of the shaft, exposing the lumen of the shaft to the exterior.

3. The applicator according to claim 1, wherein the pusher includes a hollow tube with a needle at its distal end.

4. The applicator according to claim 3, wherein the capsule is adapted to be attached to the shaft and to lock onto the distal end of the shaft.

5. The applicator according to claim 4, wherein the pusher is connected to a male Luer connector at the proximal end of the pusher.

6. The applicator according to claim 1, adapted to deliver a fillable balloon to a body cavity selected from urinary bladder, a rectum, a stomach or a vagina.

7. The applicator according to claim 1, wherein the capsule is straight.

8. The applicator according to claim 1, wherein the capsule is curved.

9. The applicator according to claim 1, adapted to treat a fistula.

10. A balloon for use in the applicator according to claim 1, having a magnet.

11. A capsule for use in the applicator according to claim 1, having a fillable balloon in an unfilled or partially filled state folded, rolled or crumpled in an interior of the capsule.

12. The capsule according to claim 11, wherein the capsule is straight.

13. The capsule according to claim 11, wherein the capsule is curved or bent.

14. The capsule according to claim 11, wherein the balloon has a magnet.

15. A system for delivering a fillable balloon into a body cavity comprising:
(a) an applicator according to claim 1; and
(b) one or more balloons configured to be folded or crumpled in the lumen of the capsule of the applicator.

16. The system according to claim 15, further comprising a retrieval device configured to deflate a filled balloon in the body cavity and to remove the balloon from the body cavity.

17. The system according to claim 16, wherein the retrieval device comprises:
(a) a needle that is movable between a retracted position, and an extended position;
(b) a plunger attached to the needle, the plunger being spring biased by a spring in the extended position; and
(c) a locking mechanism allowing the needle to be intermittently locked in the retracted position.
